(19) United States
(12) Patent Application Publication
(10) Pub. No.: US 2023/0366878 A1
(43) Pub. Date: Nov. 16, 2023

(54) METHODS OF ISOLATING AND DETECTING VIRUSES FROM LIQUID WITH POSSIBILITY OF CONTAINING VIRUSES

(71) Applicants: UNIVERSITY OF YAMANASHI, Yamanashi (JP); JNC CORPORATION, Tokyo (JP)

(72) Inventor: Eiji HARAMOTO, Yamanashi (JP)

(73) Assignees: UNIVERSITY OF YAMANASHI, Yamanashi (JP); JNC CORPORATION, Tokyo (JP)

(21) Appl. No.: 18/311,895

(22) Filed: May 4, 2023

Related U.S. Application Data
(60) Provisional application No. 63/339,977, filed on May 10, 2022.

(30) Foreign Application Priority Data
Dec. 9, 2022 (JP) 2022-197346

(51) Int. Cl. G01N 33/543

(52) U.S. Cl. CPC G01N 33/5434

(57) ABSTRACT

A method of isolating viruses from a liquid with the possibility of containing viruses, including a process of mixing a liquid with the possibility of containing viruses and a water-soluble cationic magnetic fine particle aqueous solution to form conjugates of the cationic magnetic fine particles and the viruses in a mixed solution, a process of mixing a masking agent and an aggregating agent in the mixed solution to form a magnetic composite in which the conjugates are aggregated, and a process of recovering the magnetic composite by magnetic separation, wherein the masking agent is a poly(meth)acrylic acid having a mass-average molecular weight of 5,000 to 100,000, and the poly(meth)acrylic acid is mixed in the mixed solution in a concentration range of 0.01 to 0.1 mass %.

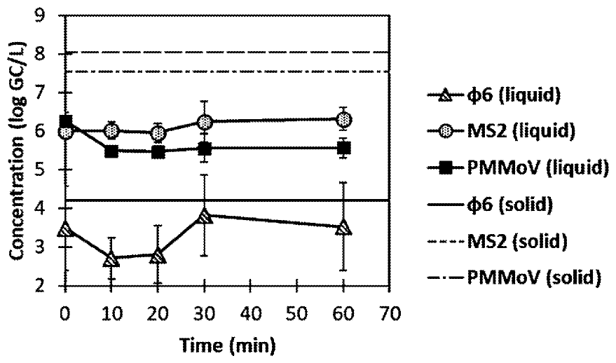

### CROSS-REFERENCE TO RELATED APPLICATION

This application claims the priority benefit of U.S. Provisional Application No. 63/339,977, filed on May 10, 2022 and Japan application serial no. 2022-197346, filed on Dec. 9, 2022. The entirety of the above-mentioned patent application is hereby incorporated by reference herein and made a part of this specification.

### BACKGROUND

### Technical Field

The disclosure relates to a method of isolating and detecting viruses from a liquid with the possibility of containing viruses, and more specifically, to a method of isolating and detecting viruses such as coronaviruses from a liquid with the possibility of containing viruses such as domestic wastewater and industrial wastewater.

### Description of Related Art

In recent years, infectious diseases caused by various viruses such as novel coronaviruses, influenza viruses, and noroviruses have occurred, and recently, an infectious disease (COVID-19) caused by a novel coronavirus (SARS-CoV-2) has spread worldwide and become an extreme problem for humankind.

In order to prevent the spread of viral infectious diseases, it is essential to detect viruses, and detection of viruses in domestic wastewater and industrial wastewater (hereinafter simply referred to as “wastewater”) discharged from general households, factories, offices and the like has been focused on. Several studies reported that SARS-CoV-2 RNA was present in the stools of individuals infected with the novel coronavirus including asymptomatic patients, and based on these findings, epidemiological studies using wastewater have been performed as a useful method for monitoring onset of COVID-19 in the community by tracking the presence of SARS-CoV-2 in wastewater (for example, refer to Non-Patent Documents 1 to 4).

In order to detect viruses in wastewater, it is important to perform analysis at a certain virus concentration, and if the virus concentration in wastewater is low, it may be impossible to detect the viruses. Therefore, the wastewater is concentrated to increase the virus concentration, and for example, methods such as adsorption using a negatively charged membrane, a negatively charged membrane vortex, aluminum hydroxide adsorption precipitation, ultrafiltration, and an ultracentrifugal method are used. For example, Non-Patent Documents 5 and 6 disclose that the polyethylene glycol (PEG) precipitation method exhibited excellent performance in concentrating SARS-CoV-2 or its surrogate viruses in wastewater.

In addition, Non-Patent Document 7 discloses the mechanism of protein precipitation by PEG, and in the PEG precipitation method, the PEG polymer is hydrated with water molecules, and its insolubility allows proteins to be concentrated as a precipitate.

### Non-Patent Documents

[Non-Patent Document 1] Warish Ahmed et al., “First confirmed detection of SARS-CoV-2 in untreated wastewater in Australia: A proof of concept for the wastewater surveillance of COVID-19 in the community”, Science of The Total Environment, Volume 728, 1 Aug. 2020, 138764
[Non-Patent Document 2] Raul Gonzalez et al., “COVID-19 surveillance in Southeastern Virginia using wastewater-based epidemiology”, Water Research, Volume 186, 1 Nov. 2020, 116296
[Non-Patent Document 3] Masaaki Kitajima et al., “SARS-CoV-2 in wastewater: State of the knowledge and research needs”, Science of The Total Environment, Volume 739, 15 Oct. 2020, 139076
[Non-Patent Document 4] David Polo et al., “Making waves: Wastewater-based epidemiology for COVID-19—approaches and challenges for surveillance and prediction”, Water Research, Volume 186, 1 Nov. 2020, 116404
[Non-Patent Document 5] Patricia Angelica Barril et al., “Evaluation of viral concentration methods for SARS-CoV-2 recovery from wastewaters”, Science of The Total Environment, Volume 756, 20 Feb. 2021, 144105
[Non-Patent Document 6] Manish Kumar et al., “First proof of the capability of wastewater surveillance for COVID-19 in India through detection of genetic material of SARS-CoV-2”, Science of The Total Environment, Volume 746, 1 Dec. 2020, 141326
[Non-Patent Document 7] Donald H. Atha et al., “Mechanism of Precipitation of Proteins by Polyethylene Glycols”, THE JOURNAL OF BIOLOGICAL CHEMISTRY, Vol. 256, No. 23. December 10, pp. 12108-12117. 1981

### SUMMARY

Concentration of wastewater by the PEG precipitation method is easy in operation, and very convenient and inexpensive, but it takes about 12 hours in order to obtain a precipitate, which is not efficient.

The disclosure provides a method of isolating and detecting viruses from a liquid with the possibility of containing viruses such as wastewater easily and efficiently.

The inventors conducted extensive studies and found that using a method of mixing water-soluble cationic magnetic fine particles to which a cationic functional group is introduced with a liquid with the possibility of containing viruses to form conjugates of the cationic magnetic fine particles and viruses in the liquid can effectively isolate the viruses from the liquid, and completed the disclosure.

That is, the disclosure includes the following configurations.

[1] A method of isolating viruses from a liquid with the possibility of containing viruses, including:
a process of mixing a liquid with the possibility of containing viruses and a water-soluble cationic magnetic fine particle aqueous solution to form conjugates of the cationic magnetic fine particles and the viruses in a mixed solution;
a process of adding a masking agent and an aggregating agent to the mixed solution to form a magnetic composite in which the conjugates are aggregated; and
a process of recovering the magnetic composite by magnetic separation,
wherein the water-soluble cationic magnetic fine particles contain a substance having a cationic functional group, a substance having a hydroxyl group and a substance having magnetism,
wherein the masking agent is a poly(meth)acrylic acid having a mass-average molecular weight of 5,000 to 100,000, and the poly(meth)acrylic acid is added to the mixed solution in a concentration range of 0.01 to 0.1 mass %.
   [2] The method of isolating viruses from a liquid with the possibility of containing viruses according to [1],
wherein the viruses are at least one selected from the group consisting of coronaviruses, influenza viruses, noroviruses and enteroviruses.
   [3] The method of isolating viruses from a liquid with the possibility of containing viruses according to [1],
wherein the viruses are SARS-CoV-2.
   [4] The method of isolating viruses from a liquid with the possibility of containing viruses according to [1],
wherein the substance having magnetism is at least one selected from among magnetite, maghemite, hematite, goethite and latex magnetic beads.
   [5] The method of isolating viruses from a liquid with the possibility of containing viruses according to [1],
wherein the substance having magnetism has an average particle size of 1 to 1,000 nm.
   [6] The method of isolating viruses from a liquid with the possibility of containing viruses according to [1],
wherein the aggregating agent is at least one selected from the group consisting of a substance having a polyalkylene glycol structure in the main chain, a substance having a polyalkylene glycol structure in a side chain and a substance having a polyglycerin structure in the main chain.
   [7] A method of detecting viruses from a liquid with the possibility of containing viruses, including:
a process of adding a nucleic acid dispersion liquid to a magnetic composite isolated by the method of isolating viruses from a liquid with the possibility of containing viruses according to any one of [1] to [6] and dispersing viruses;
a process of extracting nucleic acids of the viruses; and
a process of amplifying the nucleic acids according to a nucleic acid amplification reaction.
   [8] A test method of determining whether there are viruses in a liquid with the possibility of containing viruses using the method of detecting viruses from a liquid with the possibility of containing viruses according to [7].

### BRIEF DESCRIPTION OF THE DRAWINGS

The FIGURE is a diagram showing the results of Test Example 2 and is a graph showing a change in a virus concentration in a liquid phase over time and a virus recovery rate from a solid phase.

### DESCRIPTION OF THE EMBODIMENTS

Hereinafter, the disclosure will be described in more detail. In this specification, “(meth)acryl” means at least one selected from the group consisting of acryl and methacryl. The same applies to the term “(meth)acrylate.”

According to the method of isolating viruses from a liquid with the possibility of containing viruses of the disclosure, when viruses are bound to water-soluble cationic magnetic fine particles, the conjugates are aggregated to form a magnetic composite, and the magnetic composite is magnetically separated, it is possible to quickly and easily concentrate and isolate viruses from the liquid with the possibility of containing viruses. In addition, when a poly(meth)acrylic acid as a masking agent is added to the liquid with the possibility of containing viruses together with the water-soluble cationic magnetic fine particles, it is possible to improve the virus recovery rate.

In addition, in virus detection, since cationic magnetic fine particles do not affect virus destruction when nucleic acids of viruses are extracted, the magnetic composite isolated by the isolating method according to the disclosure can be used directly without removing the substance having magnetism that forms the magnetic composite, and thus virus detection is easy and can be automated.

<Method of Isolating Viruses from Liquid with the Possibility of Containing Viruses>

A method of isolating viruses from a liquid with the possibility of containing viruses according to the disclosure includes a process of mixing a liquid with the possibility of containing viruses and a water-soluble cationic magnetic fine particle aqueous solution to form conjugates of the cationic magnetic fine particles and the viruses in a mixed solution (conjugate forming process), a process of adding a masking agent and an aggregating agent to the mixed solution to form a magnetic composite in which conjugates of the cationic magnetic fine particles and the viruses are aggregated (magnetic composite forming process), and a process of recovering the magnetic composite by magnetic separation (magnetic composite separating process).

Here, the water-soluble cationic magnetic fine particles contain a substance having a cationic functional group, a substance having a hydroxyl group and a substance having magnetism, a poly(meth)acrylic acid having a mass-average molecular weight of 5,000 to 100,000 is used as a masking agent, and the poly(meth)acrylic acid is added to the mixed solution in a concentration range of 0.01 to 0.1 mass %.

In the disclosure, “liquid with the possibility of containing viruses” indicates sewage such as domestic wastewater and industrial wastewater discharged through sewage pipes, or dirty water in a living environment such as pool water and pond water.

Viruses are substances having a phospholipid bilayer membrane (hereinafter referred to as “phospholipid vesicles”), and are captured by cationic functional groups on the surface of the water-soluble cationic magnetic fine particles to form conjugates of the virus-cationic magnetic fine particles in the aqueous solution. In addition, when a masking agent and an aggregating agent are added, a water-insoluble magnetic composite of virus-cationic magnetic fine particles-masking agent-aggregating agent is formed. Since the magnetic composite has magnetism, magnetic separation can be performed.

Viruses to be isolated in the disclosure are not particularly limited as long as they are contained in a liquid with the possibility of containing viruses. Examples thereof include coronaviruses, influenza viruses, noroviruses, enteroviruses (for example, polioviruses), cytomegaloviruses, HIV, papillomaviruses, respiratory syncytial viruses, poliomyelitis viruses, poxviruses, measles viruses, arboviruses, coxsackieviruses, herpesviruses, hantaviruses, hepatitis viruses, Lyme disease viruses, mumps viruses, and rotaviruses.

Among these, the method of the disclosure is suitable for isolating at least one selected from the group consisting of coronaviruses, influenza viruses, noroviruses and enteroviruses from the liquid with the possibility of containing viruses, and is particularly suitable for isolation of coronaviruses.

Examples of coronaviruses include SARS-CoV-2, SARS-CoV, MERS-CoV, HCoV-229E, HCoV-NL63, HCoV-HKU1, and HCoV-OC43, and among these, the method of the disclosure is suitable for isolation of SARS-CoV-2.

(Conjugate Forming Process)

In the method of isolating viruses from a liquid with the possibility of containing viruses according to the disclosure, first, a liquid with the possibility of containing viruses and a water-soluble cationic magnetic fine particle aqueous solution are mixed, and conjugates of viruses and cationic magnetic fine particles are formed in the mixed solution.

The water-soluble cationic magnetic fine particles are particles in which a substance having a cationic functional group, a substance having a hydroxyl group and a substance having magnetism are combined by covalent bonding or physical adsorption.

The substance having magnetism is a magnetic component that can be magnetically recovered in response to an external magnetic field, and examples of magnetic components include metals such as nickel, cobalt, and iron, metal oxides such as ferrite, and latex magnetic beads in which metals or metal oxides are dispersed in polymers such as polystyrene.

The substance having magnetism (magnetic component) is, for example, magnetic fine particles, and examples thereof include magnetic metal fine particles and magnetic oxide fine particles. These magnetic fine particles may contain, as necessary, rare earth elements and transition metal elements. Examples of magnetic metal fine particles include metal fine particles such as Fe—Co, Fe—Ni, Fe—Al, Fe—Ni—Al, Fe—Co—Ni, Fe—Ni—Al—Zn, and Fe—Al—Si. Examples of magnetic oxide fine particles include iron oxide (ferrite) type ferromagnetic fine particles represented by FeOx (4/3≤x≤3/2) and ferrites in which some Fe is partially replaced with Ni or Co.

More specifically, examples of materials of magnetic fine particles include fine particles of magnetite, nickel oxide, ferrite, cobalt iron oxide, barium ferrite, carbon steel, tungsten steel, KS steel, rare earth cobalt magnet, maghemite, hematite, and goethite.

The magnetic fine particles may be subjected to a surface treatment as a preparation for introducing a substance having a hydroxyl group and a substance having a cationic functional group. Regarding the surface treatment, it is possible to perform a treatment such as a silane-based coupling treatment, a titanium-based coupling treatment, a phosphoric acid-based coupling treatment, an acid treatment with hydrochloric acid, sulfuric acid or the like, or a treatment such as an alkali treatment with sodium hydroxide or the like.

The substance having magnetism may be a magnetic component in which the surface of the magnetic fine particles is coated with a latex such as polystyrene or polymethyl acrylate or a magnetic component in which the magnetic fine particles are dispersed in latex beads (these are called latex magnetic beads).

The substance having magnetism is preferably at least one selected from among magnetite, maghemite, hematite, goethite and latex magnetic beads and more preferably magnetite or maghemite.

The substance having magnetism preferably has an average particle size of 1 nm or more. If a short time of about 30 seconds is acceptable as the time until sediment of water-soluble cationic magnetic fine particles can be confirmed, the average particle size is preferably 1 to 1,000 nm.

When the substance having magnetism is magnetic fine particles, the average particle size of the magnetic fine particles is preferably 1 to 1,000 nm, more preferably 1 to 500 nm, still more preferably 1 to 300 nm, particularly preferably 10 to 300 nm, and most preferably 30 to 150 nm in consideration of dispersibility in an aqueous solution.

When the substance having magnetism is latex magnetic beads, the average particle size of the latex magnetic beads is preferably 1 to 1,000 nm, more preferably 1 to 500 nm, and still more preferably 20 nm to 300 nm in consideration of dispersibility in an aqueous solution.

The average particle size can be measured by a dynamic light scattering method, and for example, an FPAR-1000 (product name, commercially available from Otsuka Electronics Co., Ltd.) can be used as a measurement device.

Here, it is observed that a magnetic component that is small to some extent (about 100 nm) does not respond to an external magnetic field, which is because fluctuation due to the influence of Brownian motion is greater than the response to the external magnetic field.

The shape of the substance having magnetism is not particularly limited, and may be any of a spherical shape, a needle shape, a spindle shape and an amorphous shape.

The substance having a cationic functional group is a component that converts the substance having magnetism (magnetic component) into cations in an aqueous solution. Since viruses are negatively charged, the substance having magnetism can be converted into cations, which can bind to viruses.

The substance having a cationic functional group is preferably a substance having at least one functional group selected from the group consisting of primary amino groups, secondary amino groups, tertiary amino groups, quaternary ammonium groups and guanidino groups.

Examples of substances having a cationic functional group include polyallylamine, polyvinylamine, polyethylene imine, polylysine, polyguanidine, poly(N,N-dimethylaminoethyl(meth)acrylamide), poly(N,N-dimethylaminopropyl(meth)acrylamide), polyaminopropyl(meth)acrylamide, diethylaminoethyl chloride hydrochloride, ethylenediamine, hexamethylenediamine, tris(aminoethyl)amine, aziridine hydrochloride, aminopropyltriethoxysilane, and aminoethylaminopropyltriethoxysilane. Among these, polyethylene imine or polylysine is preferable.

The substances having a cationic functional group may be used alone or two or more thereof may be used in combination.

The substance having a hydroxyl group is a component for introducing a substance having a cationic functional group into a substance having magnetism (magnetic component).

The substance having a hydroxyl group is a substance having a polyol structure. Examples of polyols include polysaccharides such as dextran, dextrin, cellulose, agarose, starch, and gellan, polysaccharide derivatives such as carboxymethyl cellulose, diethylamino cellulose, hydroxyacetyl cellulose, hydroxyacetyl cellulose, carboxymethyl dextran, diethylaminoethyl cellulose, and diethylaminoethyl dextran, synthetic polyols such as polyvinyl alcohol and polyallyl alcohol, polymers containing, as a polymerization component, at least one polymerizable monomer having a hydroxyl group such as allyl alcohol, 2-hydroxyethyl (meth)acrylate, glycerol-mono(meth)acrylate, or 2-hydroxyethyl(meth)acrylamide as a polymerizable monomer, and polyvinyl alcohol random copolymers obtained by removing protection of a hydroxyl group from polymers containing, as a polymerization component, at least one polymerizable monomer having a vinyl alcohol having a protected hydroxyl group of an acetate ester type, a trimethylsilyl ether type, or a t-butoxycarbonyloxy type. These polyols may be used alone or two or more thereof may be used in combination.

Among these, a neutral polymer having a sugar framework such as polysaccharides or polysaccharide derivatives is preferable. Specifically, any polymer may be used as long as it has an effect of forming a phase for aqueous two-phase partition, and examples thereof include water-soluble polymers having a sugar framework such as a glucose framework such as starch, and more preferably, dextran.

As the dextran, one having an optimal mass-average molecular weight can be selected through experiments and used, and for example, one having a mass-average molecular weight of 10,000 to 100,000, one having a mass-average molecular weight of 60,000 to 600,000, and also one having a mass-average molecular weight of 67,300 to 500,900 may be exemplified, and they are commercially available from, for example, Sigma-Aldrich.

Here, “aqueous two-phase partition” is a method in which an aqueous solution of two substances, for example, polyvinyl alcohol and polyethylene glycol, is mixed, and a third component is extracted using a difference in the partition coefficient between the third component and each of the formed solid layer and the aqueous solution layer without using an organic solvent.

For combining a substance having magnetism (magnetic component), a substance having a hydroxyl group and a substance having a cationic functional group, a method in which, first, a substance having magnetism and a substance having a hydroxyl group are combined to obtain water-soluble magnetic fine particles, and the water-soluble magnetic fine particles and cationic functional groups are combined to obtain water-soluble cationic magnetic fine particles may be exemplified.

Specifically, for the cationic magnetic fine particles, cationic magnetic fine particles into which a substance having a cationic functional group is introduced into a structure in which a substance having magnetism is covered with a substance having a hydroxyl group can be used.

In addition, for the cationic magnetic fine particles, cationic magnetic fine particles into which a substance having a cationic functional group is introduced into a structure in which a substance having magnetism is covered with a substance having a hydroxyl group obtained by making an acidic aqueous solution containing a polyol and metal ions alkaline can be used.

In addition, for the cationic magnetic fine particles, water-soluble cationic magnetic fine particles obtained by introducing a polyethylene imine into dextran-coated magnetite having an aldehyde obtained by treating a dextran-coated magnetite obtained by adding ammonia to an acidic aqueous solution containing dextran and iron chloride with sodium periodate by a reductive amination method can be used.

In addition, for the cationic magnetic fine particles, water-soluble cationic magnetic fine particles obtained by reacting a polyvinyl alcohol-coated magnetite having a glycidyl group obtained by treating a dextran-coated magnetite obtained by adding ammonia to an acidic aqueous solution containing polyvinyl alcohol and iron chloride with epichlorohydrin with polylysine can be used.

Examples of modes of combining a magnetic component, a substance having a hydroxyl group and a substance having a cationic functional group include physical adsorption and covalent bond formation.

Combining can be performed in an aqueous solution, and purified water, deionized water, pure water, tap water or the like can be used as the solvent.

Specifically, for water-soluble magnetic fine particles (composite of substances having a hydroxyl group-magnetic component), polyol-coated ferrite fine particles obtained by a co-precipitation method of adding an alkali such as ammonia or sodium hydroxide to an aqueous iron ion solution containing a polyol may be used (for example, refer to Japanese Patent Laid-Open No. H6-92640). More specifically, for example, as described in U.S. Pat. No. 4,452,773, particles can be obtained by a method in which a mixed aqueous solution (10 mL) of ferric chloride/hexahydrate (1.51 g) and ferrous chloride/tetrahydrate (0.64 g) is added to a dextran 50 mass % aqueous solution (10 mL) and stirred, and heating is performed in a water bath at 60 to 65° C. while a 7.4(V-V) % aqueous ammonia solution is added dropwise so that the pH becomes about 10 to 11, and the reaction occurs for 15 minutes.

In addition, as a method of combining water-soluble magnetic fine particles and cationic functional groups, specifically, according to a method in which sodium periodate (10 mg) is made to act on an dextran-coated magnetic fine particle aqueous solution (1 mass %, 100 mL) and reacted at 50° C. for 5 hours to form dextran-coated magnetic fine particles having an aldehyde group, and polyethylene imine (M. W.=1,800, 1 g) is then added to an aqueous solution dissolved in ultra-pure water (9 g), the mixture is stirred for 14 hours, the imine bond forms a dextran coating to which a polyethylene imine is bound, additionally, an aqueous solution in which sodium borohydride (10 mg) is dissolved in ultra-pure water (1 mL) is added, the mixture is stirred for 24 hours, and thus the imine bond is converted into the amine bond, polyethylene imine-introduced dextran-coated magnetic fine particles can be obtained.

As another method, an aqueous solution (1 mass %, 10 mL) containing magnetic fine particles having a glycidyl group obtained by reacting magnetic fine particles with glycidyloxypropyltriethoxysilane or by reacting polyvinyl alcohol-coated magnetic fine particles with epichlorohydrin under alkaline conditions is mixed with ε-polylysine (100 mg), and the mixture is stirred for 24 hours, and thus polylysine-introduced magnetic fine particles can be obtained.

In addition, cationic magnetic fine particles can also be obtained by reacting hydroxyl groups on the magnetic fine particles with an amine-introducing reagent such as N,N-diethylaminoethyl chloride hydrochloride (DEAE-Cl·HCl). More specifically, a 1 N aqueous sodium hydroxide solution (1 mL) and DEAE-Cl·HCl (100 mg) are added to a dextran-coated magnetic fine particle aqueous solution (1 mass %, 10 mL) and reacted for 24 hours, and thus a DEAE-introduced dextran-coated magnetic fine particle aqueous solution is obtained.

The water-soluble cationic magnetic fine particles used in the disclosure preferably have a positive charge. The charge of the water-soluble cationic magnetic fine particles can be measured as a potential, and for example, ELS-800 (product name, commercially available from Otsuka Electronics Co., Ltd.), ZetaPALS (product name, commercially available from BIC) can be used as a measurement device.

The potential ζ of the cationic magnetic fine particles is preferably 0 eV or more, more preferably +5 eV or more, still more preferably +15 eV or more, and most preferably +30 eV or more. As a qualitative property confirmation method, a method in which a cationic magnetic fine particle aqueous solution is mixed with CM Cellufine C-500-sf (product name: commercially available from JNC) and stirred, and change of a liquid from brown to colorless and transparent is confirmed can be used.

The water-soluble cationic magnetic fine particles preferably have an average particle size of 1 to 1,000 nm. The average particle size of the water-soluble cationic magnetic fine particles is substantially the same as the average particle size of the substance having magnetism (magnetic component), and a preferable range of the average particle size of the water-soluble cationic magnetic fine particle is the same as a preferable range of the average particle size of the magnetic component.

The water-soluble cationic magnetic fine particles used in the disclosure are preferably uniformly dispersed in an aqueous solution during a virus capturing operation.

When the water-soluble cationic magnetic fine particles are uniformly dispersed in an aqueous solution, even if a magnetic recovery operation is performed, the aqueous solution serves as a magnetic fluid, and magnetic recovery cannot be performed. On the other hand, if sediments are generated, the sediments are recovered by a magnetic recovery operation.

When aggregates are generated in a water-soluble cationic magnetic fine particle aqueous solution, it is preferable to perform re-dispersion according to a stirring treatment, an ultrasonic treatment or a heat treatment for use. It is desirable that the water-soluble cationic magnetic fine particles be stably and uniformly dispersed in an aqueous solution for 1 minute or longer without the occurrence of aggregation and precipitation, and it is preferable that aggregation and precipitation not occur for a period of preferably 2 weeks or longer and more preferably 6 months or longer.

In the conjugate forming process, a liquid with the possibility of containing viruses is mixed with the water-soluble cationic magnetic fine particle aqueous solution obtained above. Since the water-soluble cationic magnetic fine particles are positively charged, the viruses are adsorbed thereon, and conjugates of virus-cationic magnetic fine particles are formed in the mixed solution.

For the mixing ratio between the water-soluble cationic magnetic fine particle aqueous solution and the liquid with the possibility of containing viruses, it is preferable to mix the water-soluble cationic magnetic fine particles in the mixed solution so that the concentration is 0.10 to 1.0 mg/mL. Within the above range, conjugates of virus-cationic magnetic fine particles can be easily formed in the mixed solution. For the mixing ratio between the water-soluble cationic magnetic fine particle aqueous solution and the liquid, the cationic magnetic fine particles are mixed in the mixed solution so that the concentration is more preferably 0.10 to 0.75 mg/mL and still more preferably 0.25 to 0.50 mg/mL.

Examples of mixing methods include stirring with a magnetic stirrer, stirring with a mechanical stirrer, mixing with a vortex mixer, mixing by tapping, and mixing by pipetting, but the methods are not particularly limited thereto.

The time required for stirring depends on the stirring method, and when 60 μL of a liquid in a 1.5 mL screw cap tube is stirred using a vortex mixer, the time is 10 seconds or longer, preferably 20 seconds or longer, and more preferably 30 seconds or longer at 1,000 rpm.

(Magnetic Composite Forming Process)

Next, a masking agent and an aggregating agent are added to the mixed solution containing conjugates of virus-cationic magnetic fine particles obtained in the conjugate forming process, and the conjugates are aggregated to form a magnetic composite.

As described above, since magnetic recovery is not possible when water-soluble cationic magnetic fine particles are uniformly dispersed in an aqueous solution, when the conjugates of virus-cationic magnetic fine particles are in a dispersed state, recovery by magnetism is similarly not possible, but the conjugates are aggregated to form pellet-like masses and thus magnetic separation becomes possible.

The masking agent is a substance that neutralizes the positive charge of magnetic fine particles or converts them into negatively charged magnetic fine particles by forming an ion complex with amino groups present on the surface of water-soluble cationic magnetic fine particles. The masking agent is a substance having an acid structure or containing a salt thereof in its structure.

In the disclosure, a poly(meth)acrylic acid having a mass-average molecular weight of 5,000 to 100,000 is used as a masking agent. The mass-average molecular weight of the poly(meth)acrylic acid is preferably 10,000 to 100,000, more preferably 10,000 to 50,000, and still more preferably 25,000 to 50,000.

In addition, a poly(meth)acrylic acid having a mass-average molecular weight of 5,000 to 100,000 is added to the mixed solution in a concentration range of 0.01 to 0.1 mass %. When the concentration of the poly(meth)acrylic acid is within the above range, it is possible to efficiently isolate viruses from the liquid with the possibility of containing viruses.

In the disclosure, a masking agent other than the poly(meth)acrylic acid may be used. Examples of other masking agents include substances having an acidic functional group and salts thereof selected from among carboxylic acid, sulfuric acid, phosphoric acid and boric acid.

Specific examples of other masking agents include poly(meth)acrylic acid other than a poly(meth)acrylic acid having a mass-average molecular weight of 5,000 to 100,000, polycarboxymethylstyrene, hyaluronic acid, α-polyglutamic acid, ω-polyglutamic acid, gellan, carboxymethyl cellulose, carboxymethyldextran, polyphosphate, poly(sugar phosphate), nucleic acid, phosphoric acid, citric acid, dextran sulfate, polystyryl sulfate, and polystyryl borate.

The aggregating agent is a substance having a function of converting these substances into water-insoluble aggregates by mixing with conjugates of virus-cationic magnetic fine particles or conjugates of virus-cationic magnetic fine particle-masking agents.

Examples of aggregating agents include a substance having a polyether structure such as polyethylene glycol. In addition, alcohol compounds such as methanol, ethanol, n-propanol, and i-propanol which are organic solvents that can be mixed with water in any proportion to form a uniform solution, ketone compounds such as acetone and methyl ethyl ketone, amide compounds such as N,N-dimethylformamide, N,N-dimethylacetamide, and N-methylpyrrolidone, dimethyl sulfoxide, and 1,4- or 1,3-dioxane can also be preferably used as the aggregating agent.

Examples of substances having a polyether structure include a substance having a polyalkylene glycol structure in the main chain, a substance having a polyalkylene glycol structure in a side chain, and a substance having a polyglycerin structure in the main chain. Specific examples thereof include polyethylene glycol (PEG), polypropylene glycol, polyethylene glycol-polypropylene glycol-random copolymer, polyethylene glycol-polypropylene glycol-block copolymer, polymethoxyethoxy(meth)acrylate, poly(diethylene glycol-(meth)acrylate-methyl ether), poly(triethylene glycol-(meth)acrylate-methyl ether), poly(tetraethylene glycol-(meth)acrylate-methyl ether), poly(polyethylene glycol (meth)acrylate), and random and block copolymers thereof, and poly(glycerin-2-ethyl ether), poly(glycerin-2-diethylene glycol methyl ether), poly(glycerin-2-triethylene glycol methyl ether), poly(glycerin-2-tetraethylene glycol methyl ether), poly(glycerin-2-polyethylene glycol ether), poly(glycerin-2-polypropylene glycol ether), and poly(glycerin-2-polyethylene glycol ether)(glycerin-2-polypropylene glycol ether) copolymers.

Among these, “polyalkylene glycol” may be any one that has an effect of forming a phase for aqueous two-phase partition, and those known to form a partition phase in combination with more hydrophilic polymers or more hydrophobic polymers may be exemplified. The polyalkylene glycol is water-soluble, and the optimal one can be determined through experiments and selected for use, polyethylene glycol (PEG) and polypropylene glycol are preferable, and polyethylene glycol is more preferable. The polyethylene glycol having an optimal molecular weight can be selected through experiments and used, and for example, one having a number-average-molecular weight in a range of about 200 to 25,000, one having a number-average-molecular weight in a range of preferably about 3,000 to 20,000, more preferably about 6,000 to 15,000, and still more preferably about 8,000 to 10,000 may be exemplified, and for example, these are commercially available from Sigma-Aldrich and commercially available from FUJIFILM Wako Pure Chemical Corporation.

The amount of the aggregating agent added in the mixed solution is preferably 0.1 to 20 mass % in terms of the dry mass. Within the above range, conjugates of virus-cationic magnetic fine particles can be aggregated, and a magnetic composite that can be magnetically recovered can be formed. The amount of the aggregating agent added in the mixed solution is particularly preferably 4 to 10 mass %.

The aggregating agent may be added to the mixed solution at room temperature or may be added under ice cooling as necessary.

The aggregating agent can be used in the form of powder without change, and is preferably used as an aqueous solution, and in this case, preferably, the concentration of the aggregating agent is preferably 30 mass % or less. If the concentration is 30 mass % or less, handling becomes easy, for example, the viscosity of the aggregating agent solution does not become too high, which may be advantageous particularly when a small amount is dispensed. When it is necessary to use the aggregating agent in the form of powder, for example, when it is necessary to increase the concentration of the aggregating agent, it is desirable to use powder obtained by performing freeze-drying from water.

Examples of methods of mixing a mixed solution containing conjugates of virus-cationic magnetic fine particles with a masking agent and an aggregating agent include stirring with a magnetic stirrer, stirring with a mechanical stirrer, mixing with a vortex mixer, mixing by tapping, and mixing by pipetting, and the method is not particularly limited to these operations.

The time required for stirring depends on the stirring method, and when 80 μL of a liquid in a 1.5 mL screw cap tube is stirred using a vortex mixer, the time is 10 seconds or longer, preferably 20 seconds or longer, and more preferably 30 seconds or longer at 1,000 rpm.

(Magnetic Composite Separating Process)

The magnetic composite is isolated from the magnetic composite suspension obtained in the magnetic composite forming process by magnetic separation. The separating process may be performed at room temperature or may be performed under ice cooling as necessary.

In the disclosure, it is desirable to perform magnetic separation of the magnetic composite by providing a magnet on the side of the container containing the magnetic composite suspension. Here, examples of containers include Eppendorf tubes, screw cap tubes, and PCR tubes, but the container is not particularly limited. In addition, a structure having a liquid drain port at the bottom such as a tip of a pipette, which allows a liquid to easily go in and out, may be used. As another embodiment of the disclosure, magnetic recovery may be performed by immersing a magnet directly in the container containing the magnetic composite suspension or immersing a structure covered so that the magnet does not come into contact with the suspension in a liquid.

Magnetic recovery is completed when brown coloring derived from magnetic fine particles is no longer observed in the supernatant liquid. When the magnetic composite suspension contains 0.06 mass % of magnetic fine particles in terms of the dry weight, the time required for magnetic recovery is about 5 minutes or less. The time required for magnetic recovery can be shortened by increasing the amount of magnetic fine particles contained in the liquid containing the magnetic composite. In addition, the time required for magnetic recovery can be shortened by reducing the magnetic separation distance, specifically, by performing magnetic separation with a magnet on a container with a narrow width from the side of the container.

As another embodiment of the disclosure, using a container having a hole at the bottom, which allows a liquid to go in and out, while performing magnetic separation, when the liquid is extracted, the supernatant can be removed simultaneously with magnetic separation.

Here, in the disclosure, for aggregate removal, as described above, the supernatant may be removed while performing the magnetic separation, or after an aggregate pellet is formed, the supernatant may be carefully removed using a pipette or the like without aspirating the pellet. In this case, it is desirable to perform the supernatant removal operation under conditions for magnetic separation, and it is desirable to also remove the leaking liquid from the pellet after the supernatant is removed.

When the magnetic composite (magnetic pellet) containing viruses is recovered by the above method, viruses can be isolated from the liquid with the possibility of containing viruses.

<Method of Detecting Viruses from Liquid with the Possibility of Containing Viruses>

The disclosure also provides a method of detecting viruses using a pellet-like magnetic composite recovered by the method of isolating viruses from a liquid with the possibility of containing viruses.

The method of detecting viruses from a liquid with the possibility of containing viruses according to the disclosure includes
a process of adding a nucleic acid dispersion liquid to the magnetic composite and dispersing viruses (dispersing process),
a process of extracting nucleic acids of viruses (nucleic acid recovering process), and
a process of amplifying the nucleic acids according to a nucleic acid amplification reaction (nucleic acid amplifying process).

(Dispersing Process)

In the dispersing process, first, viruses are dispersed by adding a nucleic acid dispersion liquid to the magnetic composite isolated by the method of isolating viruses from a liquid with the possibility of containing viruses.

A known or commercially available specimen treatment reagent can be used as a nucleic acid dispersion liquid. For example, reagents such as an aqueous solution of chaotropic salts such as guanidine hydrochloride and commercially available reagents such as Buffer AVL (product name, commercially available from QIAGEN) bundled in QIAamp Viral RNA Mini QIAcube Kit (product name, commercially available from QIAGEN) and Ampdirect (product name, commercially available from Shimadzu Corporation) may be exemplified.

In the dispersion treatment, it is preferable to perform stirring strongly using a vortex mixer, ultrasonic waves or the like.

(Nucleic Acid Recovering Process)

Next, nucleic acids of viruses are extracted from the dispersion liquid obtained in the dispersing process. Viruses have a structure in which the nucleic acid is covered with a protein shell called a capsid or a protein and lipid membrane called an envelope, depending on the type of nucleic acid, and in the nucleic acid recovering process, the structure is destroyed and the nucleic acid is extracted. For the nucleic acid recovering process, conventional RNA extraction may be performed, and for example, QIAamp Viral RNA Mini QIAcube Kit (product name, commercially available from QIAGEN) is used.

Examples of nucleic acids contained in viruses include deoxyribonucleic acid (DNA) and ribonucleic acid (RNA), and either may be extracted.

(Nucleic Acid Amplifying Process)

Then, the nucleic acid obtained in the nucleic acid recovering process is amplified according to a nucleic acid amplification reaction.

As the nucleic acid amplifying method, a known method can be performed, and for example, a polymerase chain reaction (PCR) method can be used for detection.

As described above, viruses can be detected from the liquid with the possibility of containing viruses, and when this method is used, it is possible to determine the presence of viruses in the liquid, and thus the disclosure can also provide a test method for determining the presence of viruses in a liquid with the possibility of containing viruses using the virus detecting method described above.

As described above, the following configurations are disclosed in this specification.

<1> A method of isolating viruses from a liquid with the possibility of containing viruses, including:
a process of mixing a liquid with the possibility of containing viruses and a water-soluble cationic magnetic fine particle aqueous solution to form conjugates of the cationic magnetic fine particles and the viruses in a mixed solution;
a process of adding a masking agent and an aggregating agent to the mixed solution to form a magnetic composite in which the conjugates are aggregated; and
a process of recovering the magnetic composite by magnetic separation,
wherein the water-soluble cationic magnetic fine particles contain a substance having a cationic functional group, a substance having a hydroxyl group and a substance having magnetism,
wherein the masking agent is a poly(meth)acrylic acid having a mass-average molecular weight of 5,000 to 100,000, and the poly(meth)acrylic acid is added to the mixed solution in a concentration range of 0.01 to 0.1 mass %.
   <2> The method of isolating viruses from a liquid with the possibility of containing viruses according to <1>,
wherein the viruses are at least one selected from the group consisting of coronaviruses, influenza viruses, noroviruses and enteroviruses.
   <3> The method of isolating viruses from a liquid with the possibility of containing viruses according to <1> or <2>,
wherein the viruses are SARS-CoV-2.
   <4> The method of isolating viruses from a liquid with the possibility of containing viruses according to any one of <1> to <3>,
wherein the substance having magnetism is at least one selected from among magnetite, maghemite, hematite, goethite and latex magnetic beads.
   <5> The method of isolating viruses from a liquid with the possibility of containing viruses according to any one of <1> to <4>,
wherein the substance having magnetism has an average particle size of 1 to 1,000 nm.
   <6> The method of isolating viruses from a liquid with the possibility of containing viruses according to any one of <1> to <5>,
wherein the aggregating agent is at least one selected from the group consisting of a substance having a polyalkylene glycol structure in the main chain, a substance having a polyalkylene glycol structure in a side chain and a substance having a polyglycerin structure in the main chain.
   <7> A method of detecting viruses from a liquid with the possibility of containing viruses, including:
a process of adding a nucleic acid dispersion liquid to a magnetic composite isolated by the method of isolating viruses from a liquid with the possibility of containing viruses according to any one of <1> to <6> and dispersing viruses;
a process of extracting nucleic acids of the viruses; and
a process of amplifying the nucleic acids according to a nucleic acid amplification reaction.
   <8> A test method of determining whether there are viruses in a liquid with the possibility of containing viruses using the method of detecting viruses from a liquid with the possibility of containing viruses according to <7>.

### EXAMPLES

Hereinafter, the disclosure will be described in more detail with reference to examples, and the disclosure is not limited to these examples.

### (Test Example 1) 1. Collection of Sewage

From September 13 to Oct. 7, 2021, three sewage samples were collected from each of three treatment systems of a sewage treatment plant in Japan. The sewage was collected in a sterile polyethylene container, refrigerated and transported to a test room, and maintained at 4° C.

### 2. Seeding of Surrogate Viruses

Pseudomonas phage φ6 as a surrogate of enveloped viruses including SARS-CoV-2 and Coliphage MS2 as a surrogate of non-enveloped viruses were used. Pseudomonas syringae (NBRC 14084, the National Institute of Technology and Evaluation (NITE)) were used as the host strain for Pseudomonas phage φ6 (NBRC 105899, NITE). In addition, for Coliphage MS2 (ATCC 15597-B1), Salmonella Typhimurium WG49 was used as the host strain.

The initial concentrations of Pseudomonas phage φ6 strain and Coliphage MS2 strain were about 10¹⁰ PFU (plaque forming unit)/mL and 10¹¹ PFU/mL, respectively. Pseudomonas phage φ6 and Coliphage MS2 that were diluted 20-fold and 200-fold, respectively, were used.

### 3. Virus Isolation by Method of Disclosure

35 mL of the sewage was put into a 50 mL tube, 35 μL of each of the diluted solution of Pseudomonas phage φ6 and Coliphage MS2 was added, and the sample was slowly mixed using a rotator (product name, commercially available from Nichiryo Co., Ltd.) at 30 to 40 rpm for 20 to 30 minutes to obtain a sewage sample that imitates the sewage state containing viruses.

Next, 1.80 mL of dextran-coated magnetite (DM) cross-linked with polyethylene imine (PEI) (DM-PEI) (6.5 mg/mL) was added to the sewage sample so that the final concentration was 0.25 mg/mL. The mixed solution of the sewage sample and the DM-PEI was uniformly mixed by lightly shaking the tube.

NaCl, polyacrylic acid (PAAc), and polyethylene glycol (PEG) were added thereto so that the final concentration of NaCl was 0.1 M, the final concentration of PAAc was 0.021 w/v %, 0.042 w/v % and 0.084 w/v %, and the final concentration of PEG was 6.4 w/v % to prepare respective preparations. Here, PAAc was prepared by adding 0.96 to 4.16 mL of a PAAc aqueous solution (1 w/v %) of (product name, polyacrylic acid 5,000, commercially available from FUJIFILM Wako Pure Chemical Corporation) having a mass-average molecular weight of 5,000 and (product name, polyacrylic acid 25,000, commercially available from FUJIFILM Wako Pure Chemical Corporation) having a mass-average molecular weight of 25,000 so that the final concentration was 0.021 w/v %, 0.042 w/v % and 0.084 w/v %. For PEG, 6 mL of 50 w/v % of PEG (product name PEG8000, commercially available from Sigma-Aldrich) was added.

Each preparation was gently shaken and then incubated for 5 minutes to form a DM-PEI composite.

Then, the 50 mL tube containing the DM-PEI composite was set on a magnetic separator and left for about 10 to 20 minutes, and the DM-PEI composite was adsorbed on the magnet part.

Then, the supernatant was removed, and 300 μL of sterilized water (product name MilliQ water, commercially available from Merck Millipore) was added and suspended to obtain a concentrated sample.

### 4. Virus Isolation Using PEG Precipitation Method

Using the sewage sample prepared in “3. Virus isolation by method of disclosure,” 4 g of polyethylene glycol (product name PEG8000, commercially available from Sigma-Aldrich) and 2.35 g of NaCl were added to 40 mL of the sewage sample, and the final concentration of PEG was adjusted to 10 w/v %, and the final concentration of NaCl was adjusted to 1.0 M. The mixture was left at 4° C. while continuously stirring with a magnetic stirrer. It took overnight (about 24 hours) for the reaction. Then, 10,000 g of the mixed solution was centrifuged for 30 minutes. The obtained supernatant was discarded and the pellet was re-suspended in 500 μL of a phosphate buffered saline to obtain a concentrated sample.

### 5. RNA Extraction

RNA extraction was performed using the concentrated sample obtained in “3. Virus isolation by method of disclosure.”

RNA extraction was performed using a commercially available RNA extraction kit (product name QIAamp Viral RNA Mini Kit, commercially available from QIAGEN) according to the protocol.

140 μL of the concentrated sample was put into a 1.5 mL tube and incubated for 10 minutes. Then, the sample was set on a magnetic separator and left for 10 minutes, and the DM-PEI composite was adsorbed on the magnet part.

Then, the supernatant was removed, and 560 μL of a Buffer AVL (virus lysis buffer) was added to the remaining pellet and incubated for 10 minutes. Then, the 1.5 mL tube containing the suspension was set on a magnetic separator and left for 10 minutes to recover the supernatant. RNA was extracted from the supernatant to obtain 60 μL of virus RNA.

Here, separately from the above operation, RNA extraction was performed when PAAc was added before the Buffer AVL was added. That is, 140 μL of the concentrated sample was put into a 1.5 mL tube, 3 μL of a PAAc aqueous solution was added and incubated for 10 minutes, and the sample was then set on a magnetic separator and left for 10 minutes, the DM-PEI composite was adsorbed on the magnet part, the supernatant was removed, and a Buffer AVL was added to the remaining pellet for treatment.

In this case, PAAc aqueous solutions (1 w/v %) having a mass-average molecular weight of 5,000 and 25,000 were used as the PAAc aqueous solution.

### 6. RT-qPCR Detection

Quantitative reverse transcription PCR (reverse transcription-quantitative polymerase chain reaction (RT-qPCR)) was performed using the virus RNA extracted without adding a PAAc aqueous solution obtained above.

30 μL of virus RNA was reverse-transcribed using a commercially available cDNA synthesis kit (product name High-Capacity cDNA Reverse Transcription Kit, commercially available from Thermo Fisher Scientific) according to the protocol to obtain 60 μL of cDNA.

CDC-N1 and CDC-N2 assays (Centers for Disease Control and Prevention, 2020) were used in combination as qPCR assays targeting SARS-CoV-2 RNA, and the detection amount in the sample increased.

In addition to Pseudomonas phage φ6 and Coliphage MS2, Pepper mild mottle virus (PMMoV), which was the most abundant virus in sewage, was measured through RT-qPCR.

In qPCR, 2.5 μL of cDNA was mixed with 22.5 μL of a qPCR mixed solution containing 12.5 μL of a dedicated reagent for probe detection (product name Probe qPCR Mix, with UNG, commercially available from Takara Bio Inc.), 0.1 μL of each of forward primers and reverse primers (100 μM), 0.05 μL of probes (100 μM) and the remaining water for PCR, and qPCR was performed.

Heating Conditions for qPCR were as Follows. (Heating Conditions)

initial incubation at 25° C. for 10 minutes and denaturation at 95° C. for 30 seconds,

45 cycles of denaturation at 95° C. for 5 seconds, primer annealing or an elongation reaction (CDC-N1 and N2 and Pseudomonas phage φ6) at 60° C. for 30 seconds or primer annealing and an elongation reaction (PMMoV) at 60° C. for 60 seconds.

Here, for Coliphage MS2, primer annealing and an elongation reaction were performed at 56° C. for 60 seconds.

A calibration curve was created using gBlocks Gene Fragments (product name, commercially available from Integrated DNA Technologies, Inc.) (hereinafter abbreviated as gBlock) with 10-fold serial dilutions (concentration: 5.0×10⁰ to 5×10⁵ copies/reaction).

Negative controls were also included in all qPCR tests in order to confirm that there was no reagent contamination. All samples including reference products and negative controls were tested twice. A threshold cycle (Ct) value of 40 or more was considered as negative.

The results are shown in Tables 1 to 3.

TABLE 1
Detection performance of SARS-CoV-2 using PAAc
with different molecular weights

| Performance | Without | PAAc | PAAc |
|---|---|---|---|
| properties | PAAc | 5000 | 25000 |
| Number of positive | 0/4 (0) | 1/4 (25) | 4/4 (100) |
| wells/number of | | | |
| tested wells (% | | | |
| positive) | | | |
| Ct value | Not detected | 37.0 | 36.3 to 37.6 |
| Concentration (log | <3.1 | 5.6 | 5.3 to 5.8 |
| copies/L) | | | |

TABLE 2
Detection performance of SARS-CoV-2 according to
addition of PAAc in RNA extraction process

| Performance | Without | PAAc | PAAc |
|---|---|---|---|
| properties | PAAc | 5000 | 25000 |
| Number of positive | 4/4 (100) | 2/4 (50) | 0/4 (0) |
| wells/number of | | | |
| tested wells (% | | | |
| positive) | | | |
| Ct value | 36.3 to 37.6 | 35.7 | Not detected |
| Concentration (log | 5.3 to 5.8 | 5.9 | <3.1 |
| copies/L) | | | |

TABLE 3
Detection performance of SARS-CoV-2 using
PAAc with different concentrations

| | | | |
|---|---|---|---|
| Performance | 0.021% | 0.042% | 0.084% |
| properties | | | |
| Number of positive | 15/16 (94) | 16/16 (100) | 13/16 (81) |
| wells/number of | | | |
| tested wells (% | | | |
| positive) | | | |
| Ct value | 33.5 to 37.0 | 32.7 to 36.6 | 35.5 to 38.4 |
| Concentration (log | 5.1 ± 0.4 | 5.3 ± 0.4 | 4.7 ± 0.3 |
| copies/L) | | | |

(Results)

As shown in Table 1, SARS-CoV-2 was detected better when PAAc having a mass-average molecular weight of 25,000 was used in virus isolation than when PAAc having a mass-average molecular weight of 5,000 was used.

In addition, as shown in Table 2, since the detection sensitivity of SARS-CoV-2 decreased when PAAc was added in RNA extraction (virus detection), addition of PAAc in both virus isolation and RNA extraction suggested that there was some antagonistic action.

Here, as shown in Table 3, when PAAc having a mass-average molecular weight of 25,000 was added at a concentration of 0.042 w/v % in virus isolation, 100% (16/16) positive wells were obtained in qPCR, and found to be most suitable for detection of SARS-CoV-2 in sewage samples.

Based on these results, it was found that, when viruses were isolated, addition of a PAAc aqueous solution having a mass-average molecular weight of 25,000 at a concentration 0.042 w/v % was an optimal condition.

### Test Example 2

Since there was little resident SARS-CoV-2 in the sewage, Pseudomonas phage φ6 and Coliphage MS2, and PMMoV concentrations in the sewage were calculated over several time axes, and the reaction time between the virus composite and the magnet in the magnetic separator was optimized.

The preparation containing the PAAc having a final concentration of 0.042 w/v % prepared in the above “3. Virus isolation by method of disclosure” was used and gently shaken and then incubated for 5 minutes to form a DM-PEI composite.

Then, the 50 mL tube containing the DM-PEI composite was set on a magnetic separator, and 1 mL of the sample of the liquid phase portion was recovered every reaction time of 10 minutes, 20 minutes, 30 minutes, and 60 minutes.

The concentrations of Pseudomonas phage φ6, Coliphage MS2 and PMMoV in samples in a recovery liquid at each reaction time were measured using RT-qPCR. The results are shown in the FIGURE.

As shown in the FIGURE, it was found that the composite had already adhered to the magnet in the first 10 to 20 minutes. In addition, the virus concentration (counted as virus loss) in the liquid phase portion was insignificant (less than 1%) compared to the concentration of viruses recovered in the solid phase portion indicated by a horizontal straight line in the FIGURE.

It was found that the time required for virus isolation could be shortened (a maximum of 30 minutes) by performing this method.

### Test Example 3: Example 1 and Comparative Example 1

SARS-CoV-2 RNA was detected from the sewage. Detection was performed according to the detection by the method of the disclosure (Example 1) and the method by the PEG precipitation method (Comparative Example 1) for comparison.

### 1. Collection of Sewage

During 6 days from September 13 to Oct. 7, 2021, the sewage was collected from treatment systems of a sewage treatment plant in Japan. The sewage was collected in a sterile polyethylene container, refrigerated and transported to a test room, and maintained at 4° C.

### 2. Virus Isolation by Method of Disclosure (Example 1)

35 mL of the sewage was put into a 50 mL tube, subsequently, 1.8 mL of the DM-PEI (6.5 mg/mL) was added, and the tube was lightly shaken to mix the sample uniformly. The final concentration was 0.25 mg/mL.

2.04 mL of a 2 M NaCl aqueous solution, 1.96 mL of a PAAc aqueous solution (1 w/v %) having a mass-average molecular weight of 25,000, and 6.0 mL of 50 w/v % PEG (product name PEG8000, commercially available from Sigma-Aldrich) were added thereto to prepare a preparation. The final concentration of NaCl was 0.1 M, the final concentration of PAAc was 0.042 w/v %, and the final concentration of PEG was 6.4 w/v %.

The preparation was gently shaken and then incubated for 5 minutes to form a DM-PEI composite.

Then, the 50 mL tube containing the DM-PEI composite was set on a magnetic separator and left for 20 minutes, and the DM-PEI composite was adsorbed on the magnet part.

Then, the supernatant was removed, and 300 μL of sterilized water (product name MilliQ water, commercially available from Merck Millipore) was added and suspended to obtain a concentrated sample.

### 3. Virus Isolation Using PEG Precipitation Method (Comparative Example 1)

4 g of polyethylene glycol (product name PEG8000, commercially available from Sigma-Aldrich) and 2.35 g of NaCl were added to 40 mL of the sewage, and the final concentration of polyethylene glycol was adjusted to 10 w/v % and the final concentration of NaCl was adjusted to 1.0 M.

The mixture was left at 4° C. for 12 hours while continuously stirring with a magnetic stirrer. Then, 10,000 g of the mixed solution was centrifuged for 30 minutes. The obtained supernatant was discarded and the pellet was re-suspended in 500 μL of a phosphate buffered saline to obtain a concentrated sample.

### 4. RNA Extraction

RNA extraction was performed using the concentrated samples of Example 1 and Comparative Example 1.

RNA extraction was performed using a commercially available RNA extraction kit (product name QIAamp Viral RNA Mini Kit, commercially available from QIAGEN) according to the protocol.

For the concentrated sample of Example 1, 140 μL of the concentrated sample was put into a 1.5 mL tube and incubated for 10 minutes. Then, the sample was set on a magnetic separator and left for 10 minutes, and the DM-PEI composite was adsorbed on the magnet part.

Then, the supernatant was removed, and 560 μL of a Buffer AVL (virus lysis buffer) was added to the remaining pellet and incubated for 10 minutes. Then, the 1.5 mL tube containing the suspension was set on a magnetic separator and left for 10 minutes to recover the supernatant. RNA was extracted from the supernatant to obtain 60 μL of virus RNA.

For the concentrated sample of Comparative Example 1, 140 μL of the concentrated sample was dispensed, and RNA extraction was performed using a commercially available RNA extraction kit (product name QIAamp Viral RNA Mini Kit, commercially available from QIAGEN) according to the protocol to obtain 60 μL of virus RNA.

### 5. RT-qPCR Detection

Quantitative reverse transcription PCR (RT-qPCR) was performed using the virus RNA obtained above.

30 μL of virus RNA was reverse-transcribed using a commercially available cDNA synthesis kit (product name High-Capacity cDNA Reverse Transcription Kit, commercially available from Thermo Fisher Scientific) according to the protocol to obtain 60 μL of cDNA.

CDC-N1 and CDC-N2 assays (Centers for Disease Control and Prevention, 2020) were used in combination as qPCR assays targeting SARS-CoV-2 RNA, and the detection amount in the sample increased.

In addition to Pseudomonas phage φ6 and Coliphage MS2, Pepper mild mottle virus (PMMoV), which was the most abundant virus in sewage, was measured through RT-qPCR.

In qPCR, 2.5 μL of cDNA was mixed with 22.5 μL of a qPCR mixed solution containing 12.5 μL of a dedicated reagent for probe detection (product name Probe qPCR Mix, with UNG, commercially available from Takara Bio Inc.), 0.1 μL of each of forward primers and reverse primers (100 μM), 0.05 μL of probes (100 μM) and the remaining water for PCR, and qPCR was performed.

Heating conditions for qPCR were as follows. (Heating conditions)

initial incubation at 25° C. for 10 minutes and denaturation at 95° C. for 30 seconds,

45 cycles of denaturation at 95° C. for 5 seconds, primer annealing or an elongation reaction (CDC-N1 and N2 and Pseudomonas phage φ6) at 60° C. for 30 seconds or primer annealing and an elongation reaction (PMMoV) at 60° C. for 60 seconds.

Here, for Coliphage MS2, primer annealing and an elongation reaction were performed at 56° C. for 60 seconds.

A calibration curve was created using gBlocks Gene Fragments (product name, commercially available from Integrated DNA Technologies, Inc.) with 10-fold serial dilutions (concentration: 5.0×10⁰ to 5×10⁵ copies/reaction).

Negative controls were also included in all qPCR tests in order to confirm that there was no reagent contamination. All samples including reference products and negative controls were tested twice. A threshold cycle (Ct) value of 40 or more was considered as negative.

The results are shown in Table 4.

TABLE 4
Results of detection of SARS-CoV-2 from sewage

| Collection | Number of | Number of positive samples | |
|---|---|---|---|
| date | samples | | Com- |
| (day/month/ | measure- | | parative |
| year) | ment | Example 1 | Example 1 |
| 13 Sep. 2021 (*) | 2 | 1 | 1 |
| 20 Sep. 2021 (*) | 2 | 2 | 2 |
| 24 Sep. 2021 | 1 | 1 | 1 |
| 27 Sep. 2021 (*) | 2 | 0 | 1 |
| 30 Sep. 2021 | 1 | 0 | 0 |
| 7 Oct. 2021 | 1 | 0 | 0 |
| Sum | 9 | 4 (44%) | 5 (56%) |
| Ct value | — | 36.8 to 37.7 | 35.4 to 37.8 |
| Concentration | — | 4.7 to 5.0 | 4.5 to 4.8 |
| (log copies/L) | | | |

* collection of sewage from two different lines in same sewage treatment plant

As can be understood from Table 4, in Example 1, SARS-CoV-2 was successfully detected, and the detection rate was 44% assuming that SARS-CoV-2 was present at a certain concentration or more in the sewage this time. The result of Comparative Example 1 was 56%, which was slightly better, but it indicates that the method of the disclosure had performance not inferior to the PEG precipitation method.

The time required for a specimen treatment was about 30 minutes in Example 1, which was a shorter time compared to 12 hours or longer in Comparative Example 1, and thus viruses could be efficiently isolated by the method of the disclosure.

What is claimed is:

1. A method of isolating viruses from a liquid with the possibility of containing viruses, comprising:
a process of mixing a liquid with the possibility of containing viruses and a water-soluble cationic magnetic fine particles aqueous solution to form conjugates of the cationic magnetic fine particles and the viruses in a mixed solution;
a process of adding a masking agent and an aggregating agent to the mixed solution to form a magnetic composite in which the conjugates are aggregated; and
a process of recovering the magnetic composite by magnetic separation,
wherein the water-soluble cationic magnetic fine particles contain a substance having a cationic functional group, a substance having a hydroxyl group and a substance having magnetism,
wherein the masking agent is a poly(meth)acrylic acid having a mass-average molecular weight of 5,000 to 100,000, and the poly(meth)acrylic acid is added to the mixed solution in a concentration range of 0.01 to 0.1 mass %.

2. The method of isolating viruses from a liquid with the possibility of containing viruses according to claim 1,
wherein the viruses are at least one selected from the group consisting of coronaviruses, influenza viruses, noroviruses and enteroviruses.

3. The method of isolating viruses from a liquid with the possibility of containing viruses according to claim 1,
wherein the viruses are SARS-CoV-2.

4. The method of isolating viruses from a liquid with the possibility of containing viruses according to claim 1,
wherein the substance having magnetism is at least one selected from among magnetite, maghemite, hematite, goethite and latex magnetic beads.

5. The method of isolating viruses from a liquid with the possibility of containing viruses according to claim 1,
wherein the substance having magnetism has an average particle size of 1 to 1,000 nm.

6. The method of isolating viruses from a liquid with the possibility of containing viruses according to claim 1,
wherein the aggregating agent is at least one selected from the group consisting of a substance having a polyalkylene glycol structure in the main chain, a substance having a polyalkylene glycol structure in a side chain and a substance having a polyglycerin structure in the main chain.

7. A method of detecting viruses from a liquid with the possibility of containing viruses, comprising:
a process of adding a nucleic acid dispersion liquid to a magnetic composite isolated by the method of isolating viruses from a liquid with the possibility of containing viruses according to claim 1 and dispersing viruses;
a process of extracting nucleic acids of the viruses; and
a process of amplifying the nucleic acids according to a nucleic acid amplification reaction.

8. A test method of determining whether there are viruses in a liquid with the possibility of containing viruses using the method of detecting viruses from a liquid with the possibility of containing viruses according to claim 7.
